SEQUENCE LISTING (24 sequences)

SEQ ID NO: 1 - protein, 25 aa, Bacillus anthracis
SNSRKKRSTSAGPTVPDRDNDGIPD

SEQ ID NO: 2 - protein, 25 aa, Bacillus anthracis
SPEARHPLVAAYPIVHVDMENIILS

SEQ ID NO: 3 - protein, 25 aa, Bacillus anthracis
RIIFNGKDLNLVERRIAAVNPSDPL

SEQ ID NO: 4 - protein, 26 aa, Bacillus anthracis
ERIIFNGKDLNLVERRIAAVNPSDPL

SEQ ID NO: 5 - protein, 25 aa, Bacillus anthracis
RQDGKTFIDFKKYNDKLPLYISNPN

SEQ ID NO: 6 - protein, 21 aa, Bacillus anthracis
SDFEKVTGRIDKNVSPEARHP

SEQ ID NO: 7 - protein, 25 aa, Bacillus anthracis
HVDMENIILSKNEDQSTQNTDSQTR

SEQ ID NO: 8 - protein, 25 aa, Bacillus anthracis
TDSQTRTISKNTSTSRTHTSEVHGN

SEQ ID NO: 9 - protein, 26 aa, Bacillus anthracis
ETDSQTRTISKNTSTSRTHTSEVHGN

SEQ ID NO: 10 - protein, 25 aa, Bacillus anthracis
HGNAEVHASFFDIGGSVSAGFSNSN

SEQ ID NO: 11 - protein, 21 aa, Bacillus anthracis
SNSNSSTVAIDHSLSLAGERT

SEQ ID NO: 12 - protein, 18 aa, Bacillus anthracis
ETMGLNTADTARLNANIR

SEQ ID NO: 13 - protein, 24 aa, Bacillus anthracis
EPTTSLVLGKNQTLATIKAKENQE

SEQ ID NO: 14 - protein, 24 aa, Bacillus anthracis
PSKNLAPIALNAQDDFSSTPITMN

SEQ ID NO: 15 - protein, 20 aa, Bacillus anthracis
SEVLPQIQETTARIIFNGKD

SEQ ID NO: 16 - protein, 25 aa, Bacillus anthracis
NGKDLNLVERRIAAVNPSDPLETTK

SEQ ID NO: 17 - protein, 25 aa, Bacillus anthracis
ETTKPDMTLKEALKIAFGFNEPNGN

SEQ ID NO: 18 - protein, 23 aa, Bacillus anthracis
QGKDITEFDFNFDQQTSQNIKNQ

SEQ ID NO: 19 - protein, 20 aa, Bacillus anthracis
DRNNIAVGADESVVKEAHRE

SEQ ID NO: 20 - protein, 25 aa, Bacillus anthracis
REVINSSTEGLLLNIDKDIRKILSG

SEQ ID NO: 21 - protein, 19 aa, Bacillus anthracis
DMLNISSLRQDGKTFIDFK

SEQ ID NO: 22 - protein, 22 aa, Bacillus anthracis
TKENTIINPSENGDTSTNGIKK

SEQ ID NO: 23 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 24 - protein, 22 aa, Bacillus anthracis
SLLTEVETPIRNEGCRCNDSSD

(19) United States
(12) Patent Application Publication
(10) Pub. No.: US 2007/0128213 A1
(43) Pub. Date: Jun. 7, 2007

(54) Novel plant virus particles and methods of inactivation thereof

(75) Inventors: Lada Rasochova, Del Mar, CA (US); Jamie P. Phelps, Aurora, CO (US)

Correspondence Address: THE DOW CHEMICAL COMPANY, 9330 ZIONSVILLE ROAD, INDIANAPOLIS, IN, 46268, US

(73) Assignee: Dow Global Technologies Inc., Midland, MI (US)

(21) Appl. No.: 11/607,405

(22) Filed: Dec. 1, 2006

Related U.S. Application Data
(60) Provisional application No. 60/742,197, filed on Dec. 2, 2005.

(51) Int. Cl. A01H 1/00; A61K 39/12; C12N 15/82

(52) U.S. Cl. 424/199.1; 800/280; 977/802

(57) ABSTRACT

The present invention relates generally to plant viruses, produced by plants, for use as vaccines and the like. More specifically, the present invention relates to simple inactivation methods, and plant virus particles thereby obtained. The invention described herein provides means and methods to produce a safe vaccine based on an epitope display of epitopes derived from a pathogenic agent on the surface of inactivated plant virus-like particles. This invention teaches inactivation of chimeric plant virus particles and integration of the inactivation step into the virus particle purification procedure. The inactivation method renders the virus incapable of infecting plants and the integrity of virus particles is retained.

### CROSS REFERENCE TO RELATED APPLICATIONS

This application claims priority from U.S. Provisional Application Ser. No. 60/742,197, filed Dec. 2, 2005, the disclosure of which is incorporated by reference herein in its entirety.

### GOVERNMENTAL RIGHTS

This invention was made in part with government support under Grant No. 1U01AI054641-01 awarded by the National Institute of Health. The government has certain rights in this invention.

### FIELD OF THE INVENTION

The present invention relates generally to plant viruses, produced by plants, for use as vaccines and the like. More specifically, the present invention relates to virus inactivation methods and to plant virus particles as vaccines and the like.

### BACKGROUND OF THE INVENTION

Vaccination can protect individuals and entire populations from infectious agents. Developing safe and effective vaccines is, however, not always straightforward for a number of reasons ranging from identification of effective antigens to safety concerns with developed vaccines. The use of viruses as carriers of foreign peptides has been explored in the field of composite virus vaccines. Such vaccines are based on chimeric viruses, which are hybrids of different animal virus components. Usually the major component of such hybrids is derived from a virus that which is or has been rendered harmless, and the minor component is a selected antigenic component of a pathogenic virus. For example, a pox virus such as vaccinia or an attenuated poliovirus may be used as a vector for immunogenic components of other animal viruses including human viruses.

However, such techniques as discussed above can be disadvantages. Such vaccines are produced from viruses grown in cell culture systems, which can be expensive to design and run. The composite virus approach involves genetic manipulation of live, animal-infecting viruses, with the risk that mutations may give rise to novel forms of the virus with altered infectivity, antigenicity, and/or pathogenicity. In addition, the animal virus used as the vector can be a virus to which the animal may already have been exposed, and the animal may already be producing antibodies to the vector. Thus, the vector can be destroyed by the immune system before the incorporated antigenic site of the second virus induces an immune response.

A number of methods have been used for mammalian virus inactivation. These include: UV irradiation, UV/psoralen irradiation, Pentose Pharmaceuticals chemicals, Microwaves, Formalin, BPL, pH, temperature, and incubation in ammonium chloride. UV irradiation has been used to inactivate recombinant plant viruses. See e.g. Langeveld et al. (2001) “Inactivated Recombinant Plant Virus Protects Dogs from a Lethal Challenge with Canine Parvovirus,” Vaccine 19:3661-3670.

Patents that relate to methods of producing the particles and to the use of the particles, particularly as vaccines include U.S. Pat. No. 6,110,466, which discusses assembled particles of a plant virus containing a predetermined foreign peptide as part of the coat protein of the virus and U.S. Pat. No. 6,884,623 which discusses assembled particles of a plant virus containing a foreign peptide insert in the coat protein of the virus, where the site of the insert is preferably free from direct sequence repeats flanking the insert.

U.S. Pat. No. 5,602,242 relates to recombinant RNA viruses for encapsidation of genetically engineered viral sequences in heterologous, preferably rod-shaped coat, protein capsids. This patent also relates to methods of making and using such recombinant viruses, specifically with respect to the transfection of plants to bring about genotypic and phenotypic changes in the plants. Means for deleting or inactivating viral coat protein genes were described in Ahlquist et al. (1981) “Complete Nucleotide Sequence of Brome Mosaic Virus RNA3,” J. Mol. Biol. 153:23-38.

Burge et al., “Effect of Heat on Virus Inactivation by Ammonia”, Appl. Environ. Microbiology, Aug. 46(2):446-51, 1983, discusses the effect of heat on virus inactivation with ammonium chloride. Bacteriophage f2 and poliovirus 1 (an enveloped, mammalian virus) were studied. Temperatures above 40° C. were found to damage the virus tested herein. Cramer W N, et al. “Kinetics of virus inactivation by ammonia”, Appl Eniviron Microbiology, Mar 45(3):760-5, 1983, like Burge et al., used ammonium chloride, at a range of pHs, to treat sewage in an attempt to inactivate viruses. Again, bacteriophage f2 and poliovirus 1 (strain CHAT) were studied. The results of those tests are reported to show that the poliovirus inactiviation rate was influenced much less, if at all, by the effect of NH₄⁺ concentration than was the inactivation rate of f2. The paper discusses possible applications of the methodology in waste water treatment plants as a possible alternative to chlorine, particularly for members of the enterovirus group.

### BRIEF SUMMARY OF THE INVENTION

The present invention includes methods for inactivating a plant virus by administering ammonium sulfate to plant material selected from the group consisting of plants, plant tissue, plant cells and protoplasts at a pH above 8.0 to produce an inactivated virus-like particle (VLP); incubating the plant material for at least ten hours; and then harvesting the inactivated VLP from the plant material. These methods can include the incorporation of a foreign peptide into the virus. The virus can be in a non-enveloped RNA virus. The inactivated VLP can presents a heterologous bioactive peptide. The ammonium sulfate is administered at a concentration of 0.5M to 1.0M., generally at 0.7M. The pH is generally 9.0 and the plant material can be incubated at room temperature. The VLP is non-infectious because it lacks at least a portion of RNA present in the plant virus. Additionally, it can not initiate infection upon inoculation and is incapable of replicating.

The present invention can also include the inactivation of chimeric plant virus particles and integration of the inactivation step into the virus particle purification procedure. The inactivation method renders the virus incapable of infecting plants. The integrity of virus particle is maintained while the infectious viral genomic RNA that is present inside the virus particle is destroyed. These methods can be scalable and can be integrated into the purification process.

The present invention also includes methods of producing a non-infectious VLP by administering ammonium sulfate to plant material selected from the group consisting of plants, plant tissue, plant cells and protoplasts and lacks at least a portion of RNA present in a plant virus at a pH above 8.0; incubating the plant material for at least ten hours; and harvesting the inactivated VLP from the plant material, wherein the VLP is not capable of replicating.

Additionally, embodiments of the present invention can include a vaccine, wherein the vaccine includes a virus and the virus includes a foreign peptide incorporated into the virus and the vaccine is produced by a method comprising administering ammonium sulfate to plant material selected from the group consisting of plants, plant tissue, plant cells and protoplasts at a pH above 8.0 to produce an inactivated VLP; incubating the plant material for at least ten hours; and then harvesting the inactivated VLP from the plant material. The VLP peptide presented can elicit an immune response when the VLP is administered to a mammal. The vaccine can be used for influenza virus, eastern equine encephalitis virus, Canine parvovirus, or Bacillus anthracis. Additionally, the vaccine can be a subunit vaccine, wherein the peptide is a portion of an antigen and the portion is effective as a vaccine.

### BRIEF DESCRIPTION OF THE FIGURES

FIG. 1 shows RNA extracted from PA10 active and a PA10 inactivated virus run on 1.2% agarose gel stained with ethidium bromide illustrating CPMV genomic RNA 1 and 2 in the active virus and degraded RNA in the inactive virus preparation.

FIG. 2 illustrates an AIEC chromatogram of PA7E.

FIGS. 3-5 demonstrate RNA inactivation for PA9, PA11 and PA18.

FIG. 6 shows the SDS-PAGE gel of a 5 day temperature stability assay for PA1S.

FIG. 7 illustrates anti-PA antibodies in CPMV-PA immunized monkeys as detected by ELISA.

FIG. 8 shows anti-PA antibodies (IgG) in serum and bronchial lavage on day 140.

### BRIEF DESCRIPTION OF THE SEQUENCES

SEQ ID NO:1 is the peptide sequence of Epitope PA1 used according to Example 3 of the present invention.

SEQ ID NO:2 is the peptide sequence of Epitope PA2 used according to Example 3 of the present invention.

SEQ ID NO:3 is the peptide sequence of Epitope PA3 used according to Example 3 of the present invention.

SEQ ID NO:4 is the peptide sequence of Epitope PA3E used according to Example 3 of the present invention.

SEQ ID NO:5 is the peptide sequence of Epitope PA4 used according to Example 3 of the present invention.

SEQ ID NO:6 is the peptide sequence of Epitope PA5 used according to Example 3 of the present invention.

SEQ ID NO:7 is the peptide sequence of Epitope PA6 used according to Example 3 of the present invention.

SEQ ID NO:8 is the peptide sequence of Epitope PA7 used according to Example 3 of the present invention.

SEQ ID NO:9 is the peptide sequence of Epitope PA7E used according to Example 3 of the present invention.

SEQ ID NO:10 is the peptide sequence of Epitope PA8 used according to Example 3 of the present invention.

SEQ ID NO:11 is the peptide sequence of Epitope PA9 used according to Example 3 of the present invention.

SEQ ID NO:12 is the peptide sequence of Epitope PA10 used according to Example 3 of the present invention.

SEQ ID NO:13 is the peptide sequence of Epitope PA11 used according to Example 3 of the present invention.

SEQ ID NO:14 is the peptide sequence of Epitope PA12 used according to Example 3 of the present invention.

SEQ ID NO:15 is the peptide sequence of Epitope PA13 used according to Example 3 of the present invention.

SEQ ID NO:16 is the peptide sequence of Epitope PA14 used according to Example 3 of the present invention.

SEQ ID NO:17 is the peptide sequence of Epitope PA15 used according to Example 3 of the present invention.

SEQ ID NO:18 is the peptide sequence of Epitope PA16 used according to Example 3 of the present invention.

SEQ ID NO:19 is the peptide sequence of Epitope PA17 used according to Example 3 of the present invention.

SEQ ID NO:20 is the peptide sequence of Epitope PA18 used according to Example 3 of the present invention.

SEQ ID NO:21 is the peptide sequence of Epitope PA19 used according to Example 3 of the present invention.

SEQ ID NO:22 is the peptide sequence of Epitope PA20 used according to Example 3 of the present invention.

SEQ ID NO:23 is the amino acid sequence of the protective antigen (PA) of the present anthrax vaccine.

SEQ ID NO:24 is the amino acid sequence of the influenza virus epitope M2e.

### DETAILED DESCRIPTION OF THE INVENTION

The present invention will now be described more fully hereinafter with reference to the accompanying figures, in which embodiments of the invention are shown. This invention may, however, be embodied in many different forms and should not be construed as limited to the embodiments set forth herein.

The present invention relates in part to novel virus inactivation methods for making novel plant virus-like particles for use as vaccines and the like. Methods for virus inactivation are described herein. The present invention provides examples of inactivation of chimeric plant virus particles and integration of the inactivation step into the virus particle purification procedure. The inactivation method renders the virus incapable of infecting plants. Embodiments of the present invention can include means and methods to produce a safe vaccine based on an epitope display of epitopes derived from a pathogenic agent on the surface of inactivated plant virus-like particles.

Embodiments of the present invention include an efficient and scalable procedure for inactivation of viruses to produce virus-like particles (VLPs) that lack the full infectious genome of the virus. Such embodiments include using an ammonium sulfate buffer, generally at pH 9, as the initial extraction buffer. Ammonium sulfate is regarded as being non-toxic and acceptable by the regulatory authorities.

Embodiments of the present invention include viral inactivation with ammonium sulfate in a pH range of approximately 9.0. The viral inactivation can occur through RNA cleavage and degradation. The viral particles can become permeabilized allowing for the entrance of ammonium ions into the virus. Therefore, the incubation with ammonium ions should be carried out at pH above 8.0 because at this pH level the virus “swells” which “opens” its structure, thus allowing penetration of the small molecules through the viral coat.

The present invention can also provide for novel RNA virus-like particles, lacking the RNA typically associated therewith, wherein said virus-like particles comprise a properly presented antigen.

Embodiments of the present invention include methods for the integration of inactivation of particles in a seamless way with other purification operations. These methods can include instances wherein plant tissue is collected and homogenized in an extraction buffer, wherein the buffer is 0.7 M ammonium sulfate at pH 9 and incubated at room temperature for about 20 hrs. After the incubation, the particles are no longer infectious to plants and cannot initiate infection upon inoculation. The same conditions at pH 7.0 did not inactivate virus and higher temperatures i.e. 40° C. appeared to damage the virus structure.

Additional Process Steps and Parameters

Embodiments of the present invention can also include milling/homogenizing the plant material in the inactivation buffer, incubating the milled slurry in an inactivation buffer to degrade viral genomic RNA, and purifying the resulting virus particles. The milled material can be further clarified by centrifugation/filtration prior to incubation in the inactivation buffer. After the incubation, the particles can be precipitated by PEG or by increasing the molarity of ammonium sulfate to a level that causes the particle to precipitate from the solution. Buffer exchange and chromatography steps usually follow the inactivation step. The inactivation step can be integrated at any point into the purification procedure. For example, in some procedures, the inactivation was integrated into the process in a following manner: CPMV binding to HIC column in 0.7 M (NH₄)₂SO₄, pH 7 [symbol] Washing bound CPMV with 0.7 M (NH₄)₂SO₄, pH 9 [symbol] Elution of CPMV with 0.7 M (NH₄)₂SO₄, pH 9. The following ranges can be utilized: 0.5-1.0 M (NH₄)₂SO₄, pH above 8.0 and temperature between 10 to 40° C.

Types and Selection of Viruses That Can Be Used to Make VLPs

Vaccines of the present invention can be in the form of antigens and fused to coat proteins of non-enveloped RNA viruses (+, −, and/or double stranded). Embodiments of the present invention can include plant RNA viruses along with icosahedral plant RNA viruses. Although cowpea mosaic virus is exemplified herein, the methods of the present invention can be applied to other similar viruses. For example, some preferred viruses, for use according to the present invention, are:

TABLE 1

| Name | Acronym | Genus | Family |
|---|---|---|---|
| Cowpea chlorotic mottle virus | CCMV | Bromovirus | Bromoviridae |
| Cowpea mosaic virus | CPMV | Comovirus | Comoviridae |
| Tomato bushy stunt virus | TBSV | Tombusvirus | Tombusviridae |
| Alfalfa mosaic virus | AMV | Alfamovirus | Bromoviridae |
| Brome mosaic virus | BMV | Bromovirus | Bromoviridae |
| Southern bean mosaic virus | SBMV | Sobemovirus | Tombusviridae |

The present invention can be applied to any RNA plant virus. To demonstrate this system, the plant virus cowpea mosaic comovirus (CPMV) was chosen. The three-dimensional structure of the CPMV is known, which allows for identification of sites suitable for modification without disruption of the particle structure. To date, viruses from at least nine plant virus genera and three subgroup 2 ssRNA satellite viruses have had their tertiary and quaternary structures solved at high resolution. Some of these are listed above in Table 1.

One exemplified group of plant viruses for use as vectors are those whose coat proteins have a β-barrel structure. An advantage of the use of viruses that have a β-barrel structure is that the loops between the individual strands of β-sheet provide convenient sites for the insertion of foreign peptides. Modification of one or more loops can be one strategy for the expression of foreign peptides in accordance with the present invention. Insertions in other regions of the coat protein are also possible, such as insertions into the N-terminus and/or C-terminus.

All plant viruses possessing icosahedral symmetry whose structures have been solved conform to the eight stranded β-barrel fold as exemplified by CPMV, and it is likely that this represents a common structure in all icosahedral viruses. All such viruses are suitable for use in this invention for the presentation of foreign peptide sequences, which can occur in the loops between the β-strands and/or in the N-terminus and/or C-terminus.

Methods of modifying DNA sequences to insert heterologous or foreign sequences are well known to the art. Generally the viral RNA sequence is converted to a full-length cDNA transcript and cloned into a vector, then modified by inserting a foreign DNA segment in a region able to tolerate such insertion without disrupting RNA replication, particle formation, or disturbing infectivity.

Comoviruses are a group of at least fourteen plant viruses which predominantly infect legumes. Their genomes consist of two molecules of single-stranded, positive-sense RNA of different sizes which are separately encapsidated in isometric particles of approximately 28 nm diameter. The two types of nucleoprotein particles are termed middle (M) and bottom (B) component as a consequence of their behaviour in cesium chloride density gradients, the RNAs within the particles being known as M and B RNA, respectively. Both types of particle have an identical protein composition, consisting of 60 copies each of a large (VP37) and a small (VP23) coat protein. In addition to the nucleoprotein particles, comovirus preparations contain a variable amount of empty (protein-only) capsids which are known as top (T) component.

In the case of the type member of the comovirus group, cowpea mosaic virus (CPMV), it is known that both M and B RNA are polyadenylated and have a small protein (VPg) covalently linked to their 5′ terminus. More limited studies on other comoviruses suggest that these features are shared by the RNAs of all members of the group. Both RNAs from CPMV have been sequenced and shown to consist of 3481 (M) and 5889 (B) nucleotides, excluding the poly (A) tails (van Wezenbeek et al. 1983; Lomonossoff and Shanks, 1983). Both RNAs contain a single, long open reading frame. Expression of the viral gene products occurs through the synthesis and subsequent cleavage of large precursor polypeptides. Both RNAs are required for infection of whole plants. The larger B RNA is capable of independent replication in protoplasts, though no virus particles are produced in this case (Goldbach et al., 1980). This observation, coupled with earlier genetic studies, established that the coat proteins are encoded by M RNA, and the formation of infectious virus particles is dependent on the presence of both B and M viral genomic RNAs.

An advantage of the Comoviridae is that their capsid contains sixty copies each of 3 different β-barrels which can be individually manipulated. All other virus families and genera listed above have similar 3-dimensional structures but with a single type of β-barrel. (In the case of CPMV, for example, the foreign insert can be made immediately preceding the proline 23 (Pro²³) residue in the βB-βC loop of the small capsid protein (VP23). See U.S. Pat. No. 6,884,623.

The present invention can also be applied to icosahedral plant viruses (including those containing β-barrel structures) whose crystal structures have not yet been determined. Where significant sequence homology within the coat protein genes exists between one virus whose crystal structure is unknown and a second virus whose crystal structure is known, alignment of the primary structures will allow the locations of the loops between the β-strands to be inferred [see Dolja, V. V. and Koonin, E. V. (1991) J. Gen. Virol., 72, pp 1481-1486]. In addition, where a virus has only minimal coat protein sequence homology to those viruses whose crystal structure has been determined, primary structural alignments may be used in conjunction with appropriate secondary and tertiary structural prediction algorithms to allow determination of the location of potential insertion sites.

CPMV and bean pod mottle virus (BPMV) shows that the 3-D structures of BPMV and CPMV are very similar and are typical of the Comoviridae in general.

CPMV comprises two subunits, the small (S) and the large (L) coat proteins, of which there are 60 copies of each per virus particle. Foreign peptide sequences may be expressed from either the L or S proteins or from both coat proteins on the same virion.

CPMV is biparite RNA virus. In order to manipulate the genome of any RNA virus to express foreign peptides, cDNA clones of the RNA can be used. Full length cDNA clones of both CPMV RNA molecules are available, which can be manipulated to insert oligonucleotide sequences encoding a foreign peptide. cDNA clones of the genome from plant RNA viruses can be used to generate in vitro transcripts that are infectious when inoculated onto plants.

In a further aspect of the present invention, cDNA clones of CPMV RNAs M and B have been constructed, in which the cDNA clone of the M RNA contains an inserted oligonucleotide sequence encoding a foreign peptide, which make use of the cassava vein mosaic (CsVMV) promoter sequence linked to the 5′ ends of the viral cDNAs to generate infectious transcripts in the plant. This technique overcomes some of the problems encountered with the use of transcripts generated in vitro and is applicable to all plant RNA viruses.

Other viruses can include various bromoviruses, in particular the cowpea chlorotic mottle virus (CCMV) and the sobemoviruses, in particular the southern bean mosaic virus (SBMV). An RNA segment of a tripartite virus can also be used. Examples of such useful viruses are the tripartite viruses of Bromoviridae, such as brome mosaic virus (BMV) and cowpea chlorotic mottle virus (CCMV), which are packaged in icosahedral capsids.

The genome of BMV is divided among messenger sense RNA's 1, 2 and 3 of 3.2, 2.9 and 2.1 kb respectively. The coat protein is encoded by subgenomic RNA 4 that is formed from RNA3. In order for cells to be infected with BMV RNA3, the proteins encoded by BMV RNA's 1 and 2 must be present. These three BMV RNA's are separately encapsidated into identical particles. Each particle contains 180 coat protein. The coat protein can be modified to carry peptide insertions.

The coat proteins of a number of the viruses indicated in Table 1 has been compared. The similarity of the secondary structural elements and their spatial organization is illustrated in FIG. 10 of U.S. Pat. No: 6,884,623. Any of the loops that lie between the β-strands can be used for insertion of foreign epitopes. However, the insertions are made such that the additions are exposed on either the internal or external surface of the virus and such that assembly of the coat protein subunits and the infectivity of the virus are not abolished. The choice of a particular loop can be made using knowledge of the structure of individual coat protein subunits and their interactions with each other, as indicated by the crystal structure, such that any insertions are unlikely to interfere with virus assembly. The choice of precise insertion site can be made, initially, by inspection of the crystal structure, followed by in vivo experimentation to identify the optimum site.

Thus, the three dimensional structure of a plant virus can be examined in order to identify portions of a coat protein that are particularly exposed on the virus surface and are therefore potentially good sites for insertion. The amino acid sequence of the exposed portion of a coat protein can also be examined for amino acids that break α-helical structures, because these are also potentially good sites for insertion. Examples of suitable amino acids are proline and hydroxyproline, which in a polypeptide chain interrupt the α-helix and create a rigid kink or bend in the structure. N- and C-termini of coat protein are also attractive sites for insertions.

Types of Antigens and Epitopes

Embodiments of the present invention can include methods for subunit-type vaccines; that is, the presented antigen represents only a segment or segments of an antigen that is known to be effective. Such vaccines (antigens) can be inherently safer than whole organism or whole protein vaccines because they lack all functionality associated with the infective process or pathology of the disease.

Embodiments of the present invention can include methods for a subunit vaccine against the effects of anthrax (Bacillus anthracis) infection. In this anthrax vaccine, SEQ ID No: 23, the subunit antigens represent segments of about 25 amino acids derived from the so called protective antigen or PA. This protein is known to be effective in raising immunity to anthrax and is the basis for a new generation of anthrax vaccine.

Canine parvovirus vaccines can also be produced. See e.g. Langeveld et al. (2001) “Inactivated Recombinant Plant Virus Protects Dogs from a Lethal Challenge with Canine Parvovirus,” Vaccine 19:3661-3670 and Langeveld et al. (1995) “Full Protection in Mink Against Mink Enteritis Virus with New Generation Canine Parvovirus Vaccines Based on Synthetic Peptide or Recombinant Protein,” Vaccine 13:1033-1037. These viral particle-based subunit vaccines have already proven effective against a viral pathogen (Parvovirus) and protected animals from a lethal challenge with the infectious agent. The chimeric particles are currently being produced in cowpea plants by infecting the plant with pre-engineered recombinant viral RNAs or DNAs. Upon inoculation, the recombinant virus spreads cell-to-cells and long distance. This results in a systemic infection of plants. The infected plant tissue is collected, and the chimeric virus particles are extracted, formulated, and used as vaccines. According to embodiments of the present invention, it can be advantageous to inactivate the vaccine candidates to satisfy requirements for environmental protection.

The present inactivation methods can be applied not only to particles displaying antigenic epitopes that are then used as vaccines but also to particles that display any other useful peptides such as targeting peptides, antimicrobial peptides, and the like. This technology can be also applied to the wild type or modified particles that are then used for covalent linkage of various moieties to the particle surface. This includes linkage of proteins including antigenic proteins, peptides, carbohydrates, lipids, nucleic acids, detection agents (such as fluorescent dyes), radioactive agents, targeting ligands, and the like. The particle complexes can be used as vaccines as well as for delivery of the associated agents to targeted tissues and the like. This technology can be also applied prior to encapsulation of various agents, such as drugs, foreign nucleic acids for expression of foreign genes, toxins, and the like inside the particles that are then used for administration and delivery of the encapsulated agent.

Included among the many peptide epitopes that can be used according to the present invention, and expressed on the surface of the capsids, are those from viral and bacterial pathogens and cancers including those from influenza virus, eastern equine encephalitis virus, and B. anthracis.

The foreign peptide, which may be incorporated into plant viruses (see e.g. WO 92/18618), may be of highly diverse types. There may be some limitations because of the nature and size of the foreign peptide and the site at which it is placed in or on the virus particle. The peptide sequence should not interfere with the capacity of the modified virus to assemble when cultured in vivo. In this specification the term “foreign”, as applied to a peptide or to the nucleic acid encoding it, signifies peptides or nucleic acid sequences which are not native to the plant virus used as a vector. Such sequences can be alternatively described as exogenous or heterologous sequences. The term “peptide” includes small peptides and polypeptides. The peptide generally contains more than 5 amino acid residues.

Modified virus particles may be formed from any biologically useful peptides. Examples of such peptides are peptide hormones; enzymes; growth factors; antigens of protozoal, viral, bacterial, fungal or animal origin; antibodies including anti-idiotypic antibodies; immunoregulators and cytokines, e.g. interferons and interleukins; receptors; adhesins; and parts or precursors of any of the foregoing types of peptide.

Among the broad range of bioactive peptide sequences presented on plant virus vectors (in accordance with WO 92/18618, for example) special importance attaches to the antigenic peptides which are the basis of vaccines, particularly animal (including human) virus and bacterial vaccines. It should be noted that vaccines may have prophylactic (i.e. disease prevention) or therapeutic (i.e. disease treatment) applications. For vaccine applications, an especially attractive epitope presentation system is provided. When used for such applications, the antigenic peptide component will be sited appropriately on the virus particle so as to be easily recognized by the immune system, for example by location on an exposed part of the coat protein of the virus. Thus, in some embodiments of the present invention it is provided that there are assembled particles of a modified plant virus containing an antigen derived from a pathogen, e.g. an animal virus or bacterial pathogen, incorporated in an exposed position on the surface of the coat protein of the plant virus. The assembled modified plant virus particle can be used as the immunogenic component of a vaccine. Such assembled modified plant virus particles presenting antigenic peptides also have applications as the antigen presentation component of an immunodiagnostic assay for detection of, for example, animal (including human) pathogens and diseases.

In embodiments of the present invention, the antigenic VLP is inactivated and/or rendered noninfectious while maintaining the integrity of the antigens. Thus, this removes the risk of unintended transmittal of infectious viral particles, even if they are plant viruses. This can greatly reduces regulatory concerns. Thus, the transmission and spread of the plant virus to plants, after it is administered to the person or animal being treated, is greatly diminished. This system is highly versatile in regard to the size of the foreign peptide that may be inserted into the viral coat protein. Thus peptides containing up to 38 or more amino acids can be used according to the present invention.

Methods of Administration

Methods of administration for these, now recombinant, viruses can include an aerosol administered to mucous membranes. However, various methods of administration can be used according to the present invention. These include injectable administrations (IP, IM, SC), or transdermal, intranasal or oral administrations.

Candidate Viruses, Capsid Morphology Thereof, and Insertion of Antigens/Epitopes Therein

The polynucleotide segment that encodes the foreign peptide can be inserted at any suitable location in the coat protein of the original virus which does not interfere with the ability of the virus to replicate and infect the host, and which allows for proper production and presentation of the peptide on the modified virus particle. Generally, the foreign ploynucleotide is inserted so it is produced as part of or as fusion with the coat protein.

RNA transcripts are prepared, in vivo, such as in bacterial hosts, or in vitro, as known to the art, and used to inoculate an appropriate plant host or plant tissue. The RNA can be used in encapsidated form or in solution, since encapsidation will occur within the host organism. Alternatively, viral DNA fused to the DNA-dependent RNA polymerase promoter can be used to initiate the transcription of viral RNAs in vivo in the plant host. The transcribed RNA are then capable of initiating the viral infection in the plant host.

As will be understood by those skilled in the art, a given virus may require special conditions for optimal infectivity and replication, including the presence of genes acting in cis or in trans, all of which should be present when infecting the plant or plant tissue. For example, for infectivity of BMV RNA3, the presence of BMV RNA1 and 2 is necessary. Moreover, infection by a virus having the necessary host-specificity genes for a given host can in some circumstances allow infection of the host by a second virus which does not normally affect that host, e.g. mixed TMV and BMV viruses will infect both barley and tobacco even though BMV alone does not infect tobacco and TMV alone does not infect barley (Hamilton and Nichols (1977) Phytopathology, 67:484-489).

Plants may be transfected under field and/or greenhouse conditions. Abrasion of the leaf tissue is usually required for transfection. The plants can be inoculated at any time during the growth cycle, preferably when plants are young. The choice of virus and the details of modification will be matters of choice depending on parameters known and understood by those of ordinary skill in the art.

In addition to modifying the coat protein, other suitable genes may be inserted into the original viral genome for expression in the host plant. These include genes for production of commercially useful peptides, proteins, pharmaceuticals, or any other useful polypeptide in plants. In general, any heterologous gene whose expression product is functional within the plant cell can be inserted into the viral expression system described herein.

The modified coat protein itself can be inserted into a genome of a heterologous virus. In order to ensure translational fidelity of the heterologous coat protein gene, it may also be necessary to modify the translation initiation ATG codon for the original coat protein if this is not deleted, and this may be accomplished by means known to the art, such as oligonucleotide-directed substitution. If the coat protein sequence to be added has its own translational start codon, deletion or inactivation of the start codon for the original protein is necessary; alternatively, however, it may be retained and used to initiate translation of the added coat protein sequence, provided that any amino acid sequence changes introduced thereby do not interfere with RNA packaging and capsid formation.

A wide range of susceptible plant hosts and plant cells can be used. These include any dicolydenous and monocotyledonous plants, tissues of the plant as well as plant cells grown in suspension culture or forming a callus.

Further Process Steps

To produce the modified plant virus particles, the plant viral nucleic acid can be modified by introducing a nucleotide sequence coding for the foreign peptide (such as an animal virus or bacterial antigen) as a fusion with part of the plant viral genome which codes for the coat protein, infecting plants or plant cells with the modified viral nucleic acid, and harvesting assembled particles of the modified virus. The isolated viruses are then inactivated according to the present invention.

The nucleic acid sequence encoding the foreign peptide is typically introduced at the part of the plant virus genome that codes for an exposed portion of the coat protein. This procedure can be carried out by manipulation of a cDNA corresponding to the RNA of an RNA virus. In the case of an RNA virus, an RNA transcript of the modified DNA is usually prepared for inoculation of plant cells, or preferably whole plants, so as to achieve a multiplication stage prior to the harvesting of assembled particles of the modified virus. Alternatively, cDNA clones of RNA viruses may be constructed in plasmids such that 5′ ends of the viral coat protein encoding sequences are fused directly to the transcriptional start site of a promotor active in the plant host. The foreign peptide is initially expressed as part of the capsid protein and is thereby produced as part of the whole virus particle. The peptide may thus be produced as a conjugate molecule intended for use as such. Alternately, the genetic modification of the virus may be designed in order to permit release of the desired peptide from the virus particle by the application of appropriate agents which will cause cleavage from the virus particle. This may be achieved by inserting amino acid flanking the peptide of interest that are sensitive to acid hydrolysis. For example asp-pro amino acids can be engineered to flank the inserted peptide and the peptide can be released from the particle by treatment with a mild acid.

In order to produce modified virus on a commercial scale, it is not necessary to prepare ineffective inoculant (DNA or RNA transcript) for each batch of virus production. Instead, an initial inoculant may be used to infect plants; the resulting modified virus may be amplified in the plants to produce whole virus or viral RNA as inoculant for subsequent batches.

The foreign RNA or DNA may be inserted into the plant virus genome in a variety of configurations. For example, it may be inserted as an addition to the existing nucleic acid that codes for the coat protein or as a substitution for part of the existing sequence that codes for the coat protein. This choice might be determined in part by the structure of the coat protein and the ease with which additions or replacements can be made without interference with the capacity of the genetically modified virus to assemble into particles in plants. Determination of the permissible and most appropriate size of addition or deletion for the purposes of this invention may be achieved in each particular case, possibly with some additional experimentation, in the light of the present disclosure. The use of additional inserts appears to offer more flexibility than replacement inserts in some instances.

Multiplication of modified virus in plants is capable of producing significant yields. As indicated above, the inserted heterologous nucleotide sequence may include those coding for amino acids which are readily cleaved so that, after a multiplication stage, the desired material may be separated from the virus particles. For example, one could insert two peptides into the coat protein—one will be used for purification of the modified particle by, for example, affinity purification and cleaved off after purification; the other could be an antigenic peptide that will be retained on the particle and used for vaccination. As an alternative to total cleavage of the peptide, it may be possible and desirable in some cases to release the peptide in a form in which it remains intact within a major part of the capsid.

According to another aspect of the present invention, two different restriction enzyme sites may be chosen within the viral nucleic acid encoding the coat protein and the nucleic acid is restricted using the appropriate restriction enzymes. Pairs of complementary oligonucleotides are synthesized encoding the foreign peptide which it is desired to be inserted into the virus coat protein. The oligonucleotides terminate in ends which are compatible with the restriction enzymes sites thus allowing insertion into the restricted virus nucleic acid. This procedure results in the introduction of a nucleotide sequence coding for a foreign peptide into the coat protein sequence.

As used herein, the term “hybrid RNA virus” or “modified RNA virus” refers to recombinant virus RNA sequences comprising infectious viral sequences derived from an RNA virus, and a polynucleotide segment for an epitope/antigen/peptide derived from another source. Thus, prior to inactiviation, the hybrid or modified viral RNAs of this invention are RNA sequences comprising infectious viral sequences derived from one RNA virus, and a polynucleotide segment for an epitope/antigen/peptide derived from another virus, bacteria, or other sources. The term “hybrid RNA virion” or “hybrid virus particle” can be used to refer to the encapsidated form of such viruses. An original viral RNA sequence suitable for receiving an inserted peptide-encoding polynucleotide segment is an example of a sequence corresponding to that of an RNA virus. These sequences, when modified by insertion or otherwise, are “derived from” the original/naturally occurring viral sequence.

Such viral sequences must as a minimum have the functions of replicability in the host and ability to infect the host. Determinants of such functions may be required in cis or in other cases may be suppliable in trans. An example of a replication requirement satisfiable in trans is the need for the presence of the proteins encoded by BMV RNA's 1 and 2 in order to allow BMV RNA3 to replicate in a host. In contrast, certain replication signals must be present in cis (i.e. directly linked to RNA3 derivatives) to allow replication of RNA3 derivatives by the machinery induced in the infected cell by RNA's 1 and 2. Another example of trans functions are proteins encoded by CPMV RNA1 in order to allow CPMV RNA2 to replicate in a host. In contrast, certain replication signals must be present in cis (i.e. directly linked to RNA2 derivatives) to allow replication of RNA2 derivatives by the machinery induced in the infected cell by RNA1.

It can also be desirable that original viral sequences have suitable sites for the addition of foreign or heterologous peptide-encoding polynucleotides. The terms “foreign” and “heterologous” in reference to these polynucleotide segments and sequences mean sequences not in the original virus in nature. Similarly, foreign or heterologous peptide and polypeptide refer herein to the antigen or epitope that was added to the viral expression/production system. Such foreign polynucleotides or sequences may be inserted in any location not giving rise to interference with the necessary functions of the original viral sequences, i.e., the ability to replicate and infect a host. In reference to expression in a host, a “heterologous” or “isolated” polynucleotide is one which is not naturally present in the location in the host in which it has been placed. It is desirable that the placement of the heterologous peptide-encoding segments not interfere with necessary functions of the original viral sequences.

The inserted nucleic acid segments need not be naturally occurring but may be modified, composites of more than one coding segments, or encode more than one peptide/polypeptide. The RNA may also be modified by combining insertions and deletions in order to control the total length or other properties of the modified RNA molecule.

The inserted foreign RNA sequences may be non-viral or viral in origin, and may correspond either to RNA or DNA in nature. They may be prokaryotic or eukaryotic in origin, so long as they are in a form which can be directly translated by the translation machinery of the recipient cell or otherwise recognized and utilized for their functional, structural or regulatory functions.

Any plant may be infected with an RNA sequence of this invention, as will be evident to those skilled in the art, by providing appropriate host specificity and replication functions. With appropriate constructions, other eukaryotic organisms may also be infected, as may single cells and tissue cultures. This invention is not limited to any given class of host or type of RNA virus.

The term “systemic infection” means infection spread through the system of the host organism to involve more than the cells at the site of original inoculation. The entire host organism need not be infected; certain tissues can be targeted for infection. Preferred tissues are leaf tissues.

The term “transfected” as applied to the host organism means incorporation of the viral sequences of this invention into the cells of the organism in such a way as to be replicated therein. To be transfected, the organism need not be systemically infected, but can be systemically infected. However, the systemic spread of the virus is not required for the present invention.

Methods for initiating infection of the host organism are well known to the art, and any suitable method may be used. A preferred method for the infection of plants is to contact the wounded plant with a solution containing the virus or viral RNA so as to cause the virus to replicate in, or infect the plant.

Embodiments of the present invention can utilize plant viruses as vector systems for producing vaccine-like and other polypeptides in and by plants. One aspect of the present invention relates to assembled particles of a plant RNA virus containing a predetermined foreign peptide as part of the coat protein of the virus, wherein the RNA has been removed or rendered uninfectious using methods of the present invention. The present invention can also include assembled particles of a plant virus displaying a foreign peptide, wherein internal display is possible. The present invention also includes viruses that lack the infectious RNA.

As applied to the preparation of vaccines, the present invention can have advantages over conventional vaccines, recombinant vaccines based on animal viruses or bacteria, and peptide vaccines including: 1) lower production costs, as very high yields of pure virus particles are obtainable from infected plants, and no tissue culture production step is necessary; 2) improved safety, as plant viruses are incapable of infecting and replicating in animals, and thus will not be able to mutate into virulent forms, as may be the case with conventional and recombinant animal virus vaccines; 3) exceptional stability as comoviruses as purified preparations can be dried and stored for many years without losing effectiveness; 4) lack of conjugation of the peptide to the resulting in increased immunogenicity thus displaying the peptide on the surface of the particles; and 5) smaller viruses allowing for the introduction of chimeric genes by in vitro manipulation as contrasted with homologous recombination in vivo (transfection).

Unless indicated otherwise, the terms “a”, “an”, and “the” as used herein refer to at least one.

All patents, patent applications, provisional applications, and publications referred to or cited herein are incorporated by reference in their entirety to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1

### Summary of Inactivation of CPMV Particles

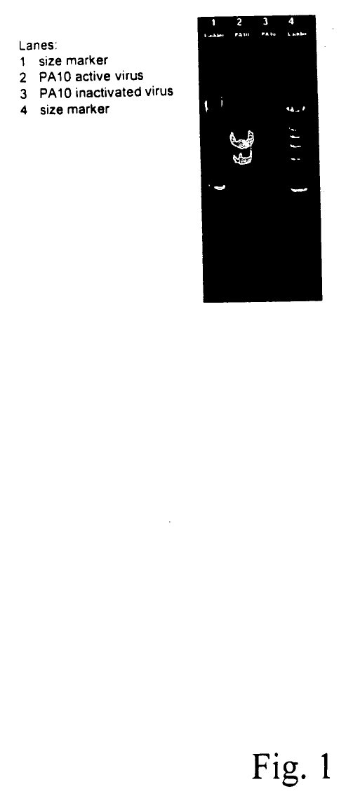

More than 16 different CPMV particles (carrying different epitopes) as well as wild type CPMV virus were inactivated using this procedure. RNA has been isolated from inactivated viruses and run on a gel to determine if the RNA is degraded. The inactivated particles have also been inoculated to plants to test for the ability to induce infection. None of the inoculated plants produced viral infection. RNA isolated from inactivated virus particles was degraded in every case tested (see FIG. 1 as an example).

### Example 2

### Further Examples of CPMV Inactivation

A study was set up to determine whether 0.8 M ammonium sulfate can inactivate CPMV while preserving its integrity. 0.8 M ammonium sulfate was used as part of the present purification process. Increase in pH during the process would permealize the virus but would also increase the concentration of free NH₃.

The conclusion from this study was that 0.8 M ammonium sulfate at pH 9 and at pH 7 both at 22° C. and 40° C. preserved virus integrity and that the virus infectivity was lost only at pH 9. The control experiments where CPMV was incubated in 30 mM Tris-HCl at 22 and 40° C. produced fully infective CPMV particles. From these results, it was further concluded that it is combination of 0.8 M ammonium sulfate and pH 9 which is required to cause inactivation and not temperature or ammonium sulfate alone. The experiments in this study were carried out on a milled cell sap adjusted to appropriate ammonium sulfate concentration and pH and there was a concern that a compound in the plant slurry was causing inactivation (e.g. psoralens). To prove or disprove this, a second study was set up to determine if purified CPMV particles can be inactivated using combination of ammonium sulfate and pH 9. Various purity grade chemicals were used to determine if some impurities in the chemicals were responsible for inactivation. An experimental matrix was set-up and all conditions tested as shown in Table 1. 0.7 M ammonium sulfate was used as it was part of our re-optimized process so that an easy integration was possible.

TABLE 2
An experimental matrix for (NH₄)₂SO₄ inactivation
study at 22° C. for 20 h.

| | Purity | Concentration | |
|---|---|---|---|
| No. | (NH₄)₂SO4 | (NH₄)₂SO4 | 30 mM Tris-HCl |
| 1 | ANALAR | 0.1 | Yes |
| 2 | ARISTAR | 0.5 | No |
| 2 | | 0.7 | |

The concentrations of 0.5 M and 0.7 M (NH₄)₂SO₄ at pH 9 inactivated CPMV while the 0.1 M (NH₄)₂SO₄ at pH 9 did not. FIG. 1 shows RNA, extracted from active and inactivated virus, run on 1.2% agarose gel and stained with EtBr. The results show presence of CPMV genomic RNA 1 and 2 in the active virus, and degraded RNA in the inactive virus preparation. The same results have been obtained for over 15 other chimeric viral particles and for the wild type virus.

### Example 3

### Chimeric CPMV Particles Used in the Inactivation Experiments

Chimeric CPMV particles were engineered to express peptides derived from the protective antigen (“PA”) protein of Bacillus anthracis. The peptides were expressed on the large and/or small coat proteins of CPMV, using the methods described in the U.S. Pat. Nos. 5,874,087, 5,958,422, and 6,110,466. The following peptides were expressed:

TABLE 3

| | | |
|---|---|---|
| Epitope | Peptide sequence | SEQ ID NO: |

| | | |
|---|---|---|
| PA1 | SNSRKKRSTSAGPTVPDRDNDGIPD | 1 |
| PA2 | SPEARHPLVAAYPIVHVDMENIILS | 2 |
| PA3 | RIIFNGKDLNLVERRIAAVNPSDPL | 3 |
| PA3E | ERIIFNGKDLNLVERRIAAVNPSDPL | 4 |
| PA4 | RQDGKTFIDFKKYNDKLPLYISNPN | 5 |
| PA5 | SDFEKVTGRIDKNVSPEARHP | 6 |
| PA6 | HVDMENIILSKNEDQSTQNTDSQTR | 7 |
| PA7 | TDSQTRTISKNTSTSRTHTSEVHGN | 8 |
| PA7E | ETDSQTRTISKNTSTSRTHTSEVHGN | 9 |
| PA8 | HGNAEVHASFFDIGGSVSAGFSNSN | 10 |
| PA9 | SNSNSSTVAIDHSLSLAGERT | 11 |
| PA10 | ETMGLNTADTARLNANIR | 12 |
| PA11 | EPTTSLVLGKNQTLATIKAKENQE | 13 |
| PA12 | PSKNLAPIALNAQDDFSSTPITMN | 14 |
| PA13 | SEVLPQIQETTARIIFNGKD | 15 |
| PA14 | NGKDLNLVERRIAAVNPSDPLETTK | 16 |
| PA15 | ETTKPDMTLKEALKIAFGFNEPNGN | 17 |
| PA16 | QGKDITEFDFNFDQQTSQNIKNQ | 18 |
| PA17 | DRNNIAVGADESVVKEAHRE | 19 |
| PA18 | REVINSSTEGLLLNIDKDIRKILSG | 20 |
| PA19 | DMLNISSLRQDGKTFIDFK | 21 |
| PA20 | TKENTIINPSENGDTSTNGIKK | 22 |

### Example 4

### Production of Chimeric CPMV Particles in Plants

Cowpea California #5 seeds from Ferry Morse, part number 1450, were germinated over night at room temperature in wet paper towels. Germinated seeds were transferred into soil. Seven days post germination the seedlings were inoculated with WT or chimeric CPMV particles. After inoculation, the plants were grown at 25° C. with a photo period of 16 hours light and 8 hours dark for two to three weeks. The leaves that showed symptoms were harvested and frozen at −80° C. prior to purification.

### Example 5

### Inactivation of Chimeric CPMV Particles and Purification of Inactivated Chimeric CPMV Virus Like Particles

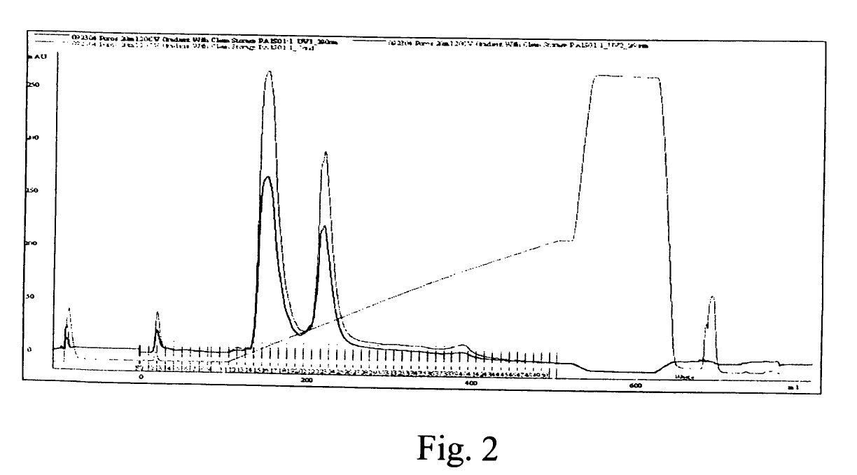

40 g of CPMV infected leaf tissue was frozen at −80° C. The frozen leaf tissue was crushed by hand and poured into a Waring high speed blender, part number 8011S. 120 ml of cold inactivation buffer (0.5M ammonium sulfate, 0.03M Tris base pH 9.00, 0.2 mM PMSF) was poured onto the crushed leaves. The leaves were ground 2 times for 3 seconds at high speed. The solution was decanted into a 500 ml centrifuge bottle. The blender was washed with 30 ml of cold inactivation buffer and the wash was poured into a 500 ml centrifuge bottle. The solution was centrifuged at 15,000 G for 30 minutes to remove the plant cellular debris. The supernatant was decanted into a graduated cylinder and incubated to inactivate the virus for 20 hours at room temperature. To precipitate the CPMV virus, cold PEG 6000 solution (20% PEG 6000, 1M NaCl) was added to the supernatant to bring the final PEG concentration to 4% PEG 6000 with 0.2M NaCl, and the solution was gently mixed. The solution was allowed to precipitate for 1 hour on ice. The virus precipitate solution was then centrifuged at 15,000 G for 30 minutes to collect the CPMV virus pellet. The supernatant was poured off and the virus was immediately resuspended in anion exchange binding buffer (30 mM Tris base, pH 7.50). To further purify the virus like particles, the protein mixture was fractionated by anion exchange chromatography using POROS 50 HQ strong anion exchange resin from Applied Biosystems, part number 1-2559-11. The 20 column volume gradient was from buffer A, 30 mM Tris base, pH 6.75, to buffer B, 30 mM Tris base, pH 6.75 with 1M NaCl. The chromatography was run with an AKTAexplorer from Amersham Biosciences, part number 18-1112-41. FIG. 2 illustrates the AIEC chromatogram of PA7E. All samples listed in the Example 3 were processed using the method described in this Example with similar results. Two major peaks were detected. The blue trace is the absorbance at 280, the red trace is the absorbance at 260, the green trace is the percent buffer B, and the brown trace is the conductivity. The red ticks on the bottom of the chromatogram are the fractions. The first peak on the gradient, which contained the desired virus like particles, was buffer exchanged into PBS buffer, pH 7.4 using a 100 kDa cutoff membrane Millipore spin concentrator, part number UFC910096. The samples were then stored at −80° C. The second peak contained the cleaved particle contaminate. An SDS-PAGE gel was prepared, with the PA7E PEG precipitate AIEC load, WT CPMV standard, and the AIEC PA7E fractions. The SDS-PAGE was ran on an Invitrogen Nupage 4-12% Bis-Tris, 12 well gel, part number NP0322. The running buffer was Invitrogen Nupage MES SDS running buffer, part number NP0002. The gel was run with a voltage drop of 200V for 35 minutes. Lane 1 contained 5 ul of the Invitrogen SeeBlue Plus2 ladder, part number LC5925. Lane 2 contained the resuspended PEG precipitate PA7E that was loaded onto the AIEC column. Lane 3 contained WT CPMV. Lanes 4-8 contained the target purified PA7E particles corresponding to the AIEC peak 1 that were collected and processed further. Lanes 9-10 contained the cleaved PA7E particles contaminate corresponding to the AIEC peak 2.

### Example 6

### Analysis of Inactivated Chimeric CPMV Virus Like Particles—Viral Genomic RNA Extraction

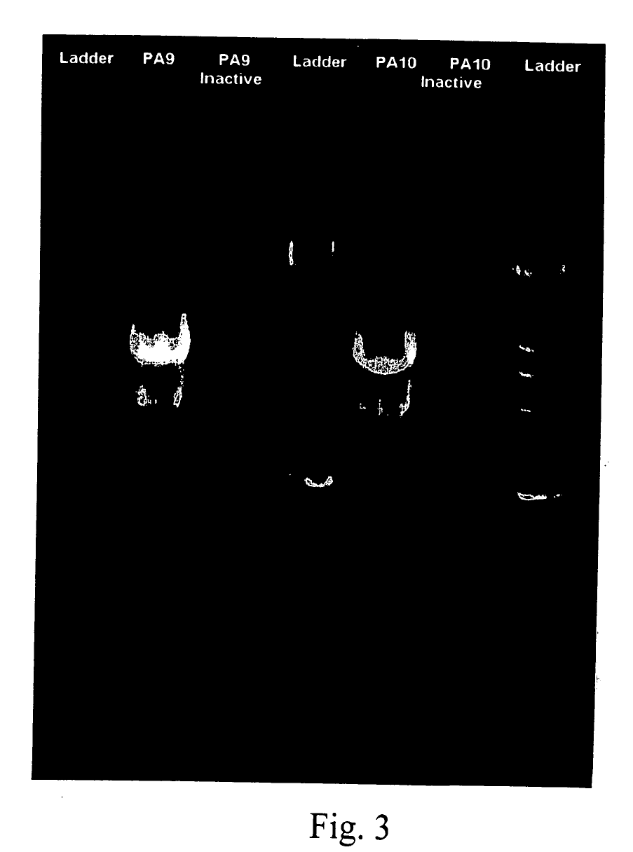

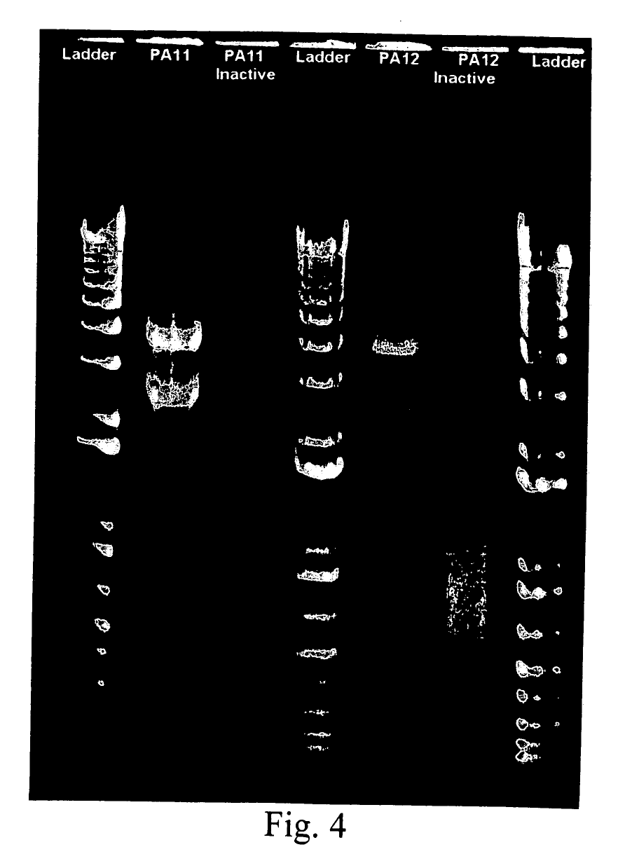

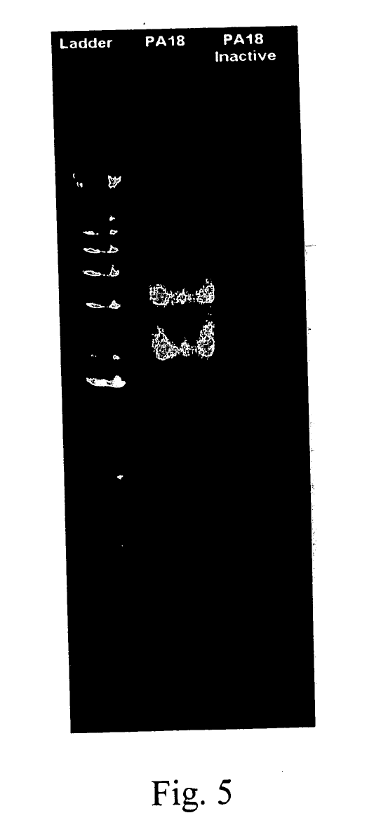

The Ambion RNAqueous, part number 1912, kit was used to extract the viral genomic RNA from the PEG purified inactivated CPMV samples. CPMV virus particles that had not been inactivated were used as a control (active samples). FIGS. 3-5 show the results of RNA inactivation for PA9, PA10, PA11, PA12, and PA18. All samples listed in the Example 3 were processed using the method described in the Example 6 with similar results. Precast 1.2% E-Gels from Invitrogen, part number G501801, were used to visualize the extracted RNA for FIGS. 1-3. All ladders in FIGS. 3-5 were 1 ul loads of 1 KB PLUS Ladder, part number 10787-026. In FIGS. 3, lane 1 is 1 ul of ladder. Lane 2 is active PA9. Lane 3 is inactivated PA9. Lane 4 is ladder. Lane 5 is active PA10, lane 6 in inactive PA10. Lane 7 is ladder. In FIGS. 4, lane 1 is 1 ul of ladder. Lane 2 is active PA1. Lane 3 is inactivated PA 11. Lane 4 is ladder. Lane 5 is active PA12, lane 6 in inactive PA12. Lane 7 is ladder. In FIGS. 5, lane 1 is 1 ul of ladder. Lane 2 is active PA18. Lane 3 is inactivated PA18. The viral genomic RNA was degraded in the inactivated samples as indicated by detection of “smear” but full-length CPMV genomic RNA1 and RNA2 was detected in samples that did not undergo inactivation.

### Example 7

### Analysis of Inactivated Chimeric CPMV Virus Like Particles—Stability by SDS-PAGE

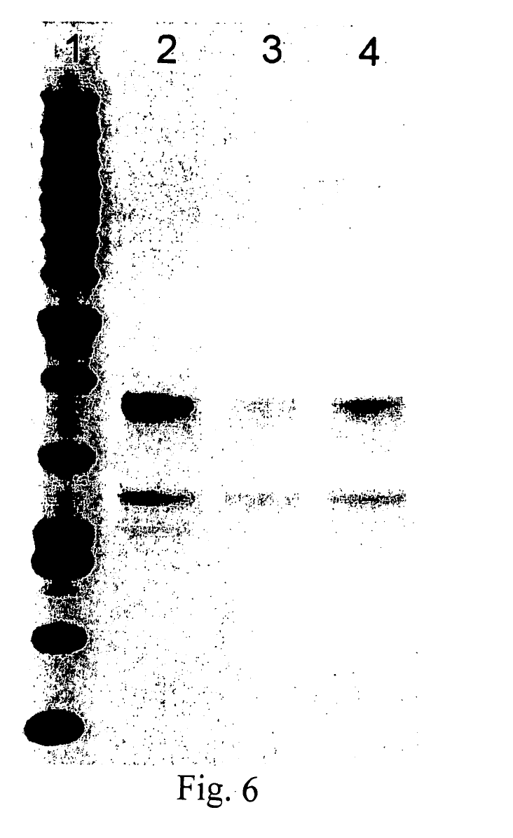

The stability of the small and large coat proteins were assayed with SDS-PAGE. FIG. 6 shows the SDS-PAGE gel of a 5 day temperature stability assay for PA1S as an example. The SDS-PAGE was ran on an Invitrogen Nupage 4-12% Bis-Tris, 12 well gel, part number NP0322. The gel was run with a voltage drop of 150V for 60 minutes. The running buffer was Invitrogen Nupage MES SDS running buffer, part number NP0002. Lane 1 contained 7 ul of Invitrogen Benchmark Unstained Protein Ladder, part number 10747-012. Lane 2 contained inactivated PA1S virus particles incubated at room temperature for 5 days. Lane 3 contained inactivated PA1S virus particles incubated at 4° C. for 5 days. Lane 4 contained inactivated PA1S virus particles incubated at −20 C for 5 days. No protein degradation was detected.

### Example 8

### Analysis of Inactivated Chimeric CPMV Virus Like Particles—Stability by SEC

The integrity of the assembled virus like particles was assayed using size exclusion chromatography (SEC). All samples listed in the Example 3 were analyzed using SEC with similar results. The SEC column used was a 30 cm×7.8 mm Tosoh TskGel G5000 analytical SEC column from Supelco with 10 micron bead size, part number 08023. The mobile phase for the SEC was 0.1M NaPO4 pH 7.00. A single peak was detected corresponding to assembled virus particles. The assembled CPMV particles eluted from the column with a retention time of 14.0 minutes.

### Example 9

### Analysis of Inactivated Chimeric CPMV Virus Like Particles—Infectivity in Plants

Inactivated chimeric CPMV particles listed in Example 3 were tested for their ability to infect plants. Cowpea California #5 seeds from Ferry Morse, part number 1450, were germinated over night at room temperature in wet paper towels. Germinated seeds were transferred into soil. Seven days post germination, ten seedlings were inoculated with inactivated and active WT or chimeric CPMV particles. After inoculation, the plants were grown at 25° C. with a photo period of 16 hours light and 8 hours dark for two to three weeks and observed for symptom formation. Plants inoculated with inactivated WT or chimeric CPMV particles showed no symptoms but plants inoculated with active WT or chimeric CPMV particles showed typical symptoms of CPMV infection. Leaves inoculated with inactivated WT or chimeric CPMV particles were harvested and processed for virus particle isolation. 40 g of leaf tissue was frozen at −80 C. The frozen leaf tissue was crushed by hand and poured into a Waring high speed blender, part number 8011S. 120 ml of cold 30 mM Tris base, pH 7.50, 0.2 mM PMSF was poured onto the crushed leaves. The leaves were ground 2 times for 3 seconds at high speed. The solution was decanted into a 500 ml centrifuge bottle. The blender was washed with 30 ml of cold buffer and the wash was poured into a 500 ml centrifuge bottle. The solution was centrifuged at 15,000 G for 30 minutes to remove the plant cellular debris. The supernatant was decanted into a graduated cylinder. To precipitate the CPMV virus, cold PEG 6000 solution (20% PEG 6000, 1M NaCl) was added to the supernatant to bring the final PEG concentration to 4% PEG 6000 with 0.2M NaCl, and the solution was gently mixed. The solution was allowed to precipitate for 1 hour on ice. The virus precipitate solution was then centrifuged at 15,000 G for 30 minutes to collect virus particles in the pellet. The supernatant was poured off and the pellet was immediately resuspended in PBS buffer, pH 7.4. The samples were assayed for the presence of virus particle with SDS-PAGE. The SDS-PAGE was ran on an Invitrogen Nupage 4-12% Bis-Tris, 12 well gel, part number NP0322. The gel was run with a voltage drop of 150V for 60 minutes. The running buffer was Invitrogen Nupage MES SDS running buffer, part number NP0002. No virus particles were detected.

### Example 10

### Immunization of Mice with Inactivated CPMV Particles Containing PA Epitope

Female Balb/c mice 7 weeks old were injected three times intraperitoneally with 100 μg purified inactivated CPMV-PA in the presence of adjuvant. Control mice received inactivated CPMV particles with unrelated peptide or only adjuvant in PBS, pH 7.0. 100 μl of Ribi adjuvant (R-700; Ribi Immunochem Research, Hamilton, Montana) mixed with 100 μl of the sample was used. Total volume for administration was 200 μl. The injections were given at 3-week intervals.

For intranasal immunization, inactivated CPMV-PA, without adjuvants, was administered to anesthetized mice. A total volume of 100 μl was administered in two nostrils (50 μl per each nostril). Control mice received inactivated CPMV with unrelated peptide or only PBS, pH 7.0.

Blood samples were obtained 1 day before the first administration and 2 weeks after each of the two subsequent administrations.

The summary of the mice immunization studies is provided below:

TABLE 4

| Adjuvant | Treatment | Route | Dose | # of mice |
|---|---|---|---|---|
| Yes | CPMV-PA | IP | 3 × 100 ug/200 ul | 5 |
| Yes | CPMV-control | IP | 3 × 100 ug/200 ul | 5 |
| Yes | PBS, pH 7.0 | IP | 3 × N/A/200 ul | 3 |
| No | CPMV-PA | IN | 3 × 100 ug/100 ul | 5 |
| No | CPMV-control | IN | 3 × 100 ug/100 ul | 5 |
| No | PBS, pH 7.0 | IN | 3 × N/A/100 ul | 3 |

### Example 11

### Immunization of Non-Human Primates with Inactivated CPMV Particles Containing PA Epitopes

The inactivated CPMV particles containing PA epitopes were tested for their ability to generate antibody responses to the co-expressed anthrax peptides when administered to rhesus macaques. Four monkeys were be immunized intramuscularly with the inactivated CPMV-PA peptide constructs and one monkey with the inactivated wild type CPMV control. Each immunizing dose consisted of 2 mg of the virus-peptide mixture of all 16 PA-CPMV constructs. The animals were vaccinated at days 0, 7, 14, and 28.

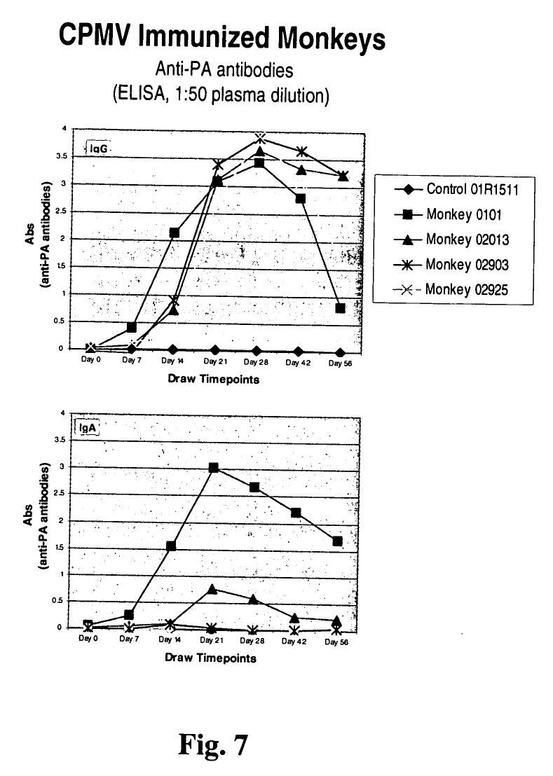

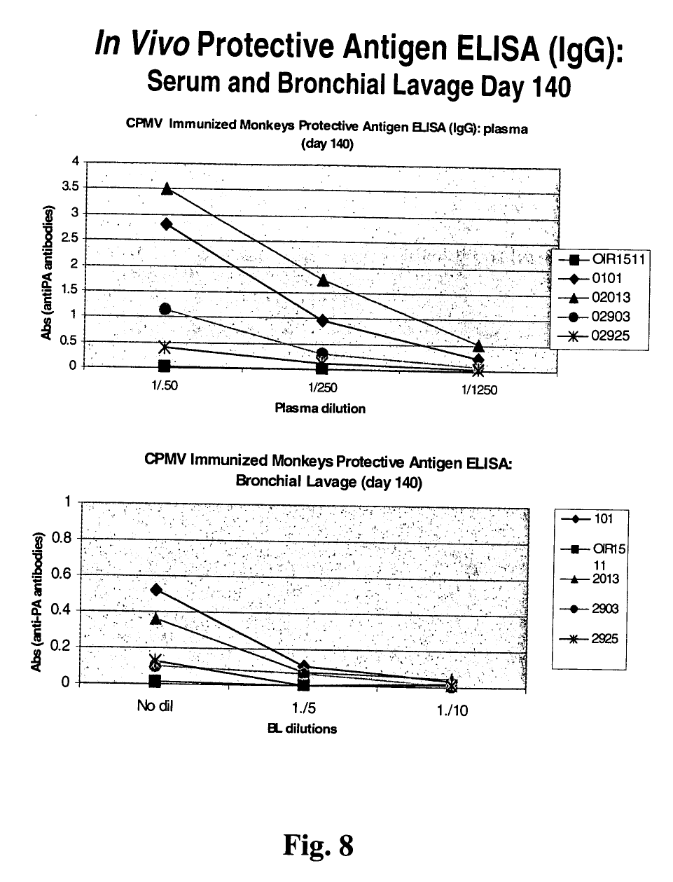

IgG and IgA antibodies were monitored using ELISA assays with PA protein as a target. Three to 5 ml of blood were drawn in heparin on each of the immunization days. Cells and plasma were separated and cryopreserved. Ketamine anesthesized monkeys were bronchoscoped on days 0, 14, and 28 and bronchial lavage specimens obtained and cryopreserved. The bronchial washings and plasma were thawed and IgG and IgA antibody titers measured in ELISA assays. High titres of both the IgG and IgA antibodies were detected in plasma and bronchial lavage. The results are shown in FIG. 7 and 8.

### Example 12

### Immunization of Mice with Inactivated CPMV Particles Containing Influenza Virus Epitope M2e

Female Balb/c mice 7 weeks old were injected three times intraperitoneally with 100 μg purified inactivated CPMV expressing an influenza peptide M2e in the presence of adjuvant. The sequence is SLLTEVETPIRNEGCRCNDSSD (SEQ ID NO: 24). Control mice received inactivated CPMV particles with unrelated peptide or only adjuvant in PBS, pH 7.0. 100 μl of Ribi adjuvant (R-700; Ribi Immunochem Research, Hamilton, Montana) mixed with 100 μl of the sample was used. Total volume for administration was 200 μl. The injections were given at 3-week intervals.

For intranasal immunization, inactivated CPMV containing an influenza peptide M2e, without adjuvants, were administered to anesthetized mice. A total volume of 100 μl was be administered in two nostrils (50 μl per each nostril). Control mice received inactivated CPMV with unrelated peptide or only PBS, pH 7.0.

Blood samples were obtained 1 day before the first administration and 2 weeks after each of the two subsequent administrations.

The summary of the mice immunization studies is provided below:

TABLE 6

| Adjuvant | Treatment | Route | Dose | # of mice |
|---|---|---|---|---|
| Yes | CPMV-M2e | IP | 3 × 100 ug/200 ul | 5 |
| Yes | CPMV-control | IP | 3 × 100 ug/200 ul | 5 |
| Yes | PBS, pH 7.0 | IP | 3 × N/A/200 ul | 3 |
| No | CPMV-M2e | IN | 3 × 100 ug/100 ul | 5 |
| No | CPMV-control | IN | 3 × 100 ug/100 ul | 5 |
| No | PBS, pH 7.0 | IN | 3 × N/A/100 ul | 3 |

The foregoing is illustrative of the present invention, and is not to be construed as limiting thereof. The invention is defined by the following claims, with equivalents of the claims to be included therein.

1. A method of inactivating a plant virus comprising:
administering ammonium sulfate to plant material expressing a virus-like particle wherein the plant material is selected from the group consisting of plants, plant tissue, plant cells and protoplasts at a pH above 8.0;
incubating the plant material for at least ten hours to produce an inactivated virus-like particle (VLP); and
harvesting the inactivated VLP from the plant material.

2. The method according to claim 1, further comprising at least one foreign peptide incorporated into the virus.

3. The method according to claim 1, wherein said virus in a non-enveloped RNA virus.

4. The method according to claim 1, wherein said inactivated VLP presents a heterologous bioactive peptide.

5. The method according to claim 1, wherein said peptide is an antigen.

6. The method according to claim 1, wherein said peptide is an epitope.

7. The method according to claim 1, wherein the ammonium sulfate is administered at a concentration of 0.5M to 1.0M.

8. The method according to claim 1, wherein the pH is pH 9.0.

9. The method according to claim 1, wherein the plant material is incubated between 10° C. to 40° C.

10. The method according to claim 2, wherein said method comprises binding said plant virus to a hydrophobic interaction chromatography column in 0.7 M (NH₄)₂SO₄ at pH 7, washing bound virus with 0.7 M (NH₄)₂SO₄ at pH 9, and eluting said virus with 0.7 M (NH₄)₂SO₄ at pH 9.

11. The method according to claim 1, wherein the virus has a capsid that is icosahedral.

12. The method according to claim 1, wherein the virus is of a family selected from the group consisting of Bromoviridae, Comoviridae, and Tombusviridae.

13. The method according to claim 1, wherein the virus is of a genus selected from the group consisting of Bromovirus, Comovirus, Tombusvirus, Alfamovirus, and Sobemovirus.

14. The method according to claim 1, wherein the virus is selected from the group consisting of cowpea mosaic virus, cowpea chlorotic mottle virus, tomato bushy stunt virus, alfalfa mosaic virus, brome mosaic virus, and southern bean mosaic virus.

15. The method according to claim 1, wherein the virus comprises coat proteins and the peptides are antigen fused to the coat proteins.

16. The method according to claim 1, wherein the peptide is selected from the group consisting of a peptide hormone, an enzyme, a growth factor, an antibody, an immunoregulator, and a cytokine.

17. The method according to claim 3, wherein said method further comprises converting a viral RNA sequence into a full-length cDNA transcript, cloning said cDNA into a vector, and modifying said cDNA by inserting a foreign DNA segment in a region able to tolerate such insertion without disrupting RNA replication, particle formation, or infectivity.

18. The method according to claim 1, wherein the foreign peptide incorporated into the virus is selected from the group consisting of a subunit of influenza virus, eastern equine encephalitis virus, Canine parvovirus, and Bacillus anthracis.

19. A method of producing a non-infectious VLP comprising:
administering ammonium sulfate to plant material selected from the group consisting of plants, plant tissue, plant cells and protoplasts and lacks at least a portion of RNA present in a plant virus at a pH above 8.0;
incubating the plant material for at least ten hours; and
harvesting the inactivated VLP from the plant material, wherein said VLP is not capable of replicating.

20. A vaccine comprising a VLP wherein said vaccine comprises a plant virus wherein said virus comprises at least one foreign peptide incorporated into the virus and the vaccine is produced by a method comprising administering ammonium sulfate to plant material selected from the group consisting of plants, plant tissue, plant cells and protoplasts at a pH above 8.0 to produce an inactivated VLP; incubating the plant material for at least ten hours; and harvesting the inactivated VLP from the plant material.

21. The vaccine of claim 20, wherein the VLP peptide presented elicits an immune response when said VLP is administered to a mammal.

22. The vaccine of claim 20, wherein the vaccine is for influenza virus, eastern equine encephalitis virus, Canine parvovirus, or Bacillus anthracis.

23. The vaccine of claim 20, wherein the peptide is an epitope.

24. The vaccine of claim 23, wherein the epitope is a viral pathogen, a bacterial pathogen, or cancer.

25. The vaccine of claim 20, wherein said vaccine is a subunit vaccine, wherein said peptide is a portion of an antigen and said portion is effective as a vaccine.

26. The vaccine of claim 20, wherein said foreign peptide comprises SEQ ID NO: 23.
